(19) United States
(12) Patent Application Publication
(10) Pub. No.: US 2026/0301971 A1
(43) Pub. Date: Oct. 1, 2026

(54) DATA GENERATION DEVICE, DATA GENERATION METHOD, AND RECORDING MEDIUM

(71) Applicant: NEC Corporation, Tokyo (JP)

(72) Inventors: Masanori TSUJIKAWA, Tokyo (JP); Remi Numajiri, Tokyo (JP); Satoshi Oda, Tokyo (JP); Masahiro Kubo, Tokyo (JP); Tomoko Morita, Tokyo (JP)

(73) Assignee: NEC Corporation, Tokyo (JP)

(21) Appl. No.: 19/567,604

(22) Filed: Mar. 16, 2026

(30) Foreign Application Priority Data
Mar. 28, 2025 (JP) 2025-054982

(51) Int. Cl. G16H 50/70; G16H 10/60

(52) U.S. Cl. CPC G16H 50/70; G16H 10/60

(57) ABSTRACT

A data generation device includes an extraction unit, a generation unit, and an output unit. The extraction unit extracts information about a medical practice to be introduced into a treatment for an injury or disease from predetermined medical information as new information. The generation unit generates, as time series medical information, information indicating a time series action to be taken for a patient regarding a medical practice to be introduced, based on the extracted new information. The output unit outputs the generated time series medical information. With such a configuration, the data generation device can support decision making regarding the treatment of a patient.

This application is based upon and claims the benefit of priority from Japanese Patent Application No. 2025-54982, filed on Mar. 28, 2025, the disclosure of which is incorporated herein in its entirety by reference.

### TECHNICAL FIELD

The present disclosure relates to a data generation device and the like.

### BACKGROUND ART

In a medical institution, time series information indicating a plan of a treatment, a test, and the like performed on a patient may be used. For example, a clinical path is used as time series information indicating a plan of a treatment, a test, and the like performed on a patient. An information system that supports the creation may be used to create the clinical path.

The clinical path creation support device of JP 2015-197733 A narrows down the candidates of the event that can be executed for the patient based on the name and attribute of the injury or disease of the patient. The clinical path creation support device of JP 2015-197733 A displays event candidates on a clinical path creation screen.

### SUMMARY

An object of the present disclosure is to provide a data generation device and the like capable of easily generating information indicating an action to be taken for a patient in a medical practice to be introduced in time series.

A data generation device according to an aspect of the present disclosure includes an extraction unit that extracts information about a medical practice to be introduced into a treatment for an injury or disease from predetermined medical information as new information, a generation unit that generates, as time series medical information, information indicating a time series action to be taken for a patient regarding a medical practice to be introduced based on the extracted new information, and an output unit that outputs the generated time series medical information.

A data generation method according to an aspect of the present disclosure includes extracting information about a medical practice introduced into a treatment for an injury or disease from predetermined medical information as new information, generating, as time series medical information, information indicating a time series action to be taken for a patient regarding a medical practice to be introduced based on the extracted new information, and outputting the generated time series medical information.

A non-transitory recording medium according to an aspect of the present disclosure records a program for causing a computer to execute the steps of extracting information about a medical practice introduced into a treatment for an injury or disease from predetermined medical information as new information, generating, as time series medical information, information indicating a time series action to be taken for a patient regarding a medical practice to be introduced based on the extracted new information, and outputting the generated time series medical information.

### BRIEF DESCRIPTION OF THE DRAWINGS

Exemplary features and advantages of the present disclosure will become apparent from the following detailed description when taken with the accompanying drawings in which:

FIG. 1 is a diagram illustrating an example of a configuration of a data generation system in the present disclosure;

FIG. 2 is a diagram illustrating an example of a configuration of the data generation device in the present disclosure;

FIG. 3 is a diagram illustrating an example of time series medical information in the present disclosure;

FIG. 4 is a diagram schematically illustrating an example of a process of generating time series medical information in the present disclosure;

FIG. 5 is a diagram schematically illustrating an example of a process of generating time series medical information in the present disclosure;

FIG. 6 is a diagram illustrating an example of an operation flow of the data generation device in the present disclosure; and

FIG. 7 is a diagram illustrating an example of a hardware configuration of the data generation device in the present disclosure.

### EXAMPLE EMBODIMENT

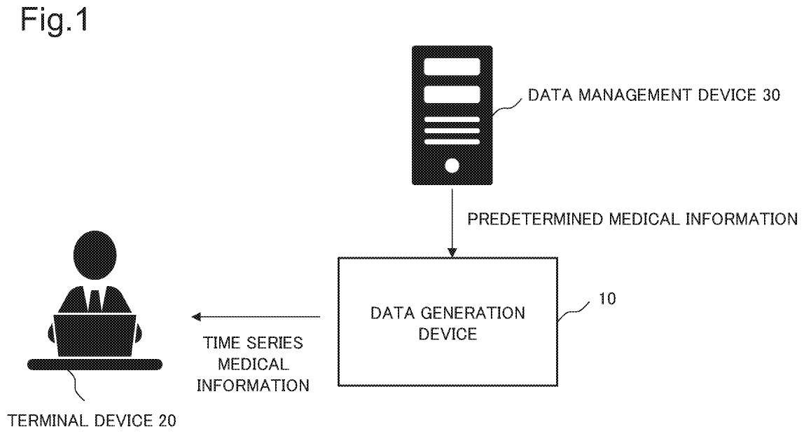

Example embodiments of the present disclosure will be described in detail with reference to the drawings. FIG. 1 is a diagram illustrating an example of a configuration of a data generation system. The data generation system includes a data generation device 10, a terminal device 20, and a data management device 30. The data generation device 10 is connected to the terminal device 20 via, for example, a network. The data generation device 10 is connected to the data management device 30 via, for example, a network. A plurality of terminal devices 20 and a plurality of data management devices 30 may be provided. The number of the terminal devices 20 and the number of the data management devices 30 can be appropriately set.

The data generation system generates, for example, information indicating a time series action taken for a patient regarding a medical practice to be introduced as time series medical information. The data generation system extracts, for example, information about a medical practice introduced into a treatment for an injury or disease from predetermined medical information as new information. The data generation system generates, for example, information indicating a time series action taken for a patient regarding a medical practice to be introduced as time series medical information based on the extracted new information. For example, the data generation system generates time series medical information using a language model based on the extracted new information. A specific example of the language model will be described later.

The medical practice to be introduced is, for example, a medical practice newly introduced by a medical institution. For example, the medical practice to be introduced is administration of a medicine to be newly introduced into a medical institution, use of a medical device, a treatment, or a surgery. The medical practice to be introduced is not limited to the above. The predetermined medical information is, for example, information describing a medical practice to be introduced. For example, the predetermined medical information is information including information of one or a plurality of items among a procedure, an effect, a caution, a usage, and an application criterion of a medical practice to be introduced. For example, the predetermined medical information is information described in a package insert of a medicine or information described in a paper. The predetermined medical information is not limited to the above. The new information is information that is required to be referred to by a medical practitioner in a medical institution in order to take an action for a patient among predetermined medical information. For example, when the medical practice to be introduced is administration of a new medicine, the new information is information about a subject to be administered with the new medicine, an administration method, and a side effect. The new information in a case where the medical practice to be introduced is prescription of a new medicine is not limited to the above.

The information indicating a time series action taken for the patient is, for example, information indicating the content of an action for treatment performed on the patient by a medical practitioner in a medical institution. The treatment may include a test for prevention of an injury or disease and confirmation of the health condition. The action for treatment performed on a patient by a medical practitioner in a medical institution is, for example, an action related to one or more of a clinical examination, a surgery, a test, a medication, a rehabilitation, a meal provision, a guidance, and a counseling. The medical practice taken for a patient by a medical practitioner in a medical institution is not limited to the above. The medical practitioner is, for example, a doctor, a nurse, a pharmacist, a clinical laboratory technician, a physical therapist, a dietitian, or a counselor. The medical practitioner is not limited to the above.

The time series medical information is information indicating a plan of an action related to the treatment performed by a medical practitioner on a patient in a medical institution. For example, the time series medical information is information indicating the order of the medical practice taken for a patient by a medical practitioner in a medical institution or the timing of taking the action. The time series medical information is, for example, a clinical path. The time series medical information may be information indicating procedures or cautions in the action taken for a patient by a medical practitioner in a medical institution. The time series medical information is not limited to the above.

The time series medical information is, for example, information viewed by a medical practitioner. The time series medical information may be information presented to the patient. The subject who views the time series medical information is not limited to the above. The time series medical information may be generated according to the role of the person who views the time series medical information. For example, the time series medical information may be generated according to medical roles, such as for the doctor and for the nurse. The time series medical information may be generated for the patient. A person whose time series medical information is to be viewed can be appropriately set.

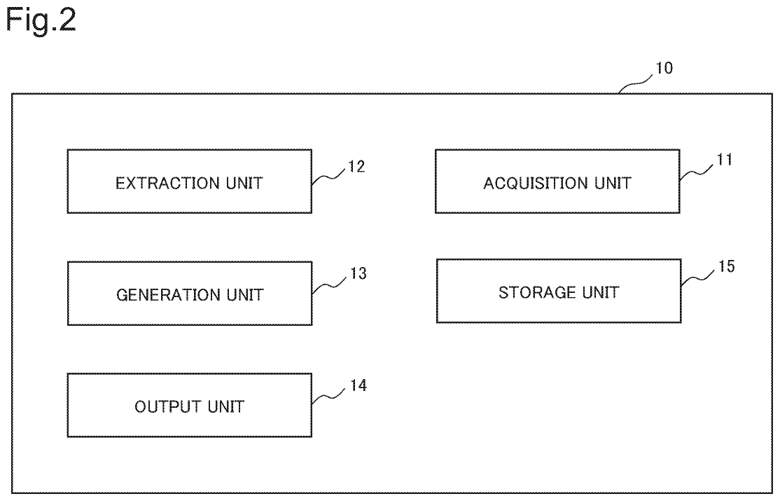

A configuration of the data generation device 10 will be described. FIG. 2 illustrates an example of a configuration of the data generation device 10. The data generation device 10 includes an extraction unit 12, a generation unit 13, and an output unit 14 as a basic configuration. The data generation device 10 further includes, for example, an acquisition unit 11 and a storage unit 15.

The acquisition unit 11 acquires, for example, predetermined medical information. The acquisition unit 11 acquires information about a medical practice to be newly introduced in a medical institution as predetermined medical information. In a case where the medical practice to be introduced is administration of a medicine to be newly introduced, the acquisition unit 11 acquires, for example, a package insert of the medicine to be newly introduced, or a paper, a report, proceedings, or a preprint described regarding the medicine, as predetermined medical information. In a case where the medical practice to be introduced is administration of a medicine to be newly introduced, the acquisition unit 11 may acquire, for example, a structural formula of the medicine to be newly introduced. In a case where the medical practice to be introduced is a therapy or a surgery to be newly introduced, the acquisition unit 11 may acquire a paper, a report, proceedings, or a preprint describing the medical practice to be introduced as the predetermined medical information. In a case where the medical practice to be introduced is use of a new medical device, the acquisition unit 11 acquires a manual of the medical device or a paper, a report, proceedings, or a preprint described regarding the medical device as predetermined medical information. The predetermined medical information acquired by the acquisition unit 11 is not limited to the above. The acquisition unit 11 acquires predetermined medical information from the data management device 30, for example. The acquisition unit 11 may acquire predetermined medical information from the terminal device 20. The acquisition unit 11 may acquire predetermined medical information from an information processing device for information provision connected via a network.

The acquisition unit 11 may acquire information about a medical practice that is the same in a target as the medical practice to be introduced is performed. “Same” may include “similar”. For example, the acquisition unit 11 acquires information about a medical practice that is the same in a target as the medical practice to be introduced among the medical practices performed in the medical institution. The acquisition unit 11 acquires time series medical information or information corresponding to predetermined medical information in a medical practice that is the same in a target as the medical practice to be introduced. The acquisition unit 11 may acquire information indicating an action to be taken for the medical practice that for a target that is the same as a target for which the medical practice to be introduced is performed. For example, in a case where the medical practice to be introduced is administration of a new medicine, the acquisition unit 11 acquires information about a method of administering a medicine that is for an injury or disease same as an injury or disease for which the new medicine to be introduced in the medical institution is administered. In a case where the medical practice to be introduced is administration of a new medicine, the acquisition unit 11 acquires information about a method of administering a medicine that is for a symptom same as a symptom for which the new medicine to be introduced in the medical institution is administered. The acquisition unit 11 may acquire information about a medical practice similar to the medical practice to be introduced. For example, the acquisition unit 11 acquires time series medical information in a medical practice to be introduced or information corresponding to predetermined medical information.

For example, the acquisition unit 11 acquires, from the data of the electronic medical record, information about the medical practice for a target that is the same as a target for which the medical practice to be introduced is performed. For example, the acquisition unit 11 acquires, from the data of the electronic medical record, information about a medical practice performed for an injury or disease that is the same as an injury or disease for which the medical practice to be introduced is to be performed. “Same” may include “similar”. For example, the acquisition unit 11 acquires, from the data of the electronic medical record stored in the data management device 30, data of the electronic medical record for a person who has received the medical practice for a target that is the same as a target for which the medical practice to be introduced is performed.

For example, the acquisition unit 11 acquires the target name of the injury or disease to which the time series medical information is applied. The target name of the injury or disease to which the time series medical information is applied is input to the terminal device 20 by an operation of a person in charge who generates the time series medical information, for example. The acquisition unit 11 acquires the target name of the injury or disease to which the time series medical information is applied from the terminal device 20, for example. The acquisition unit 11 may acquire information about a person who views the time series medical information. For example, the acquisition unit 11 acquires information indicating whether the person who views the time series medical information is a medical practitioner or a patient. The acquisition unit 11 may acquire information indicating a role in the treatment of a person who views the time series medical information. The information about the person who views the time series medical information is input to the terminal device 20 by the operation of the person in charge, for example. The acquisition unit 11 acquires information about a person who views the time series medical information from the terminal device 20, for example.

The extraction unit 12 extracts information about a medical practice to be introduced into a treatment for an injury or disease from predetermined medical information as new information. The extraction unit 12 extracts, as new information, information indicating a characteristic action in the medical practice to be introduced among actions taken for a patient for a treatment for an injury or disease in the introduced medical practice. The extraction unit 12 may extract, as the new information, information about an action having a difference from a medical practice that is the same in a target as the medical practice to be introduced is performed, among actions to be taken for a patient for a treatment for an injury or disease in the medical practice to be introduced. The extraction unit 12 may extract, as new information, information about an action that changes depending on a medical practice to be introduced, among actions to be taken for a patient for an action for an injury or disease, from predetermined medical information. For example, the extraction unit 12 extracts, from the predetermined medical information, information about an action that changes from the action in the existing medical practice by replacing the existing medical practice with the medical practice to be introduced, among actions taken for a patient for a treatment for an injury or disease. The extraction unit 12 may extract, from the predetermined medical information, information about an action that can be newly caused by the medical practice to be introduced.

The extraction unit 12 extracts new information from predetermined medical information using, for example, a language model. The language model is, for example, a machine learning model that acquires a prompt as an input, generates an answer to the prompt, and outputs a generation result. That is, the language model is, for example, a machine learning model that receives a prompt as an input to output an answer to the prompt.

For example, the extraction unit 12 inputs, to the language model, a prompt including predetermined medical information and a request for extracting information indicating a characteristic action to be taken for the patient from the predetermined medical information. The extraction unit 12 extracts, for example, information indicating a characteristic action to be taken for a patient, the information being extracted from predetermined medical information, the information being output from the language model, as new information. For example, in a case where the medical practice to be introduced is administration of a new medicine, the extraction unit 12 extracts the new information, for example, by inputting a prompt including a package insert of the new medicine and a request for extracting information indicating a characteristic action to be taken for the patient from the package insert into the language model.

For example, a machine learning model also referred to as a large-scale language model can be used as the language model. For example, Generative Pre-trained Transformer-2 (GPT-2), GPT-3, GPT-3.5, or GPT-4 can be used as the language model. Claude3, Claude3.5, a text-to-text transfer transformer (T5), bidirectional encoder representations from transformers (BERT), robustly optimized BERT approach (RoBERTa), or efficiently learning an encoder that classifies token replacements accurately (ELECTRA) may be used as the language model. The language model used for the process of extracting the new information from the predetermined medical information is not limited to the above.

The extraction unit 12 may extract the new information using a language model that operates in an information processing device outside the data generation device 10. In a case where the language model that operates in the external information processing device is used, for example, the extraction unit 12 outputs a prompt including predetermined medical information and a request for extracting information indicating a characteristic action to be taken for the patient from the predetermined medical information to the information processing device in which the language model operates. The extraction unit 12 acquires, for example, new information extracted by the language model from the information processing device in which the language model operates.

The extraction unit 12 may extract information of an item set as an extraction target item from predetermined medical information as new information. The extraction target item is, for example, information indicating an item that is required to be extracted in generating the time series medical information. The extraction target item is set so that, for example, an action that requires attention of a medical practitioner in introducing a medical practice and an item of a symptom of a patient caused by the action are extracted. The extraction target item may be generated by a person in charge of generating the time series medical information. For example, the extraction unit 12 may extract new information from predetermined medical information using an extraction model. The extraction model is, for example, a machine learning model that extracts information of an extraction target item as new information from predetermined medical information. The extraction model is generated, for example, by learning a relationship between predetermined medical information and extraction target items, and new information.

The extraction unit 12 may extract, from the predetermined medical information, a caution regarding the condition of the patient caused by performing the medical practice on the patient as new information. For example, in a case where the action to be introduced is administration of a new medicine, the extraction unit 12 extracts information about the timing at which the side effect due to the medicine may occur and the symptom. For example, when extracting new information using a language model, the extraction unit 12 extracts the new information by inputting a prompt including a package insert of a new medicine and a request for extracting information indicating a caution regarding the patient's condition from the package insert into the language model.

The extraction unit 12 may extract information about interaction between a medicine to be introduced and a medicine used for a treatment for an injury or disease as new information. For example, the extraction unit 12 refers to a database that stores data of medicine interaction and extracts information indicating interaction with another medicine as new information. The extraction unit 12 may extract information about interaction between a medicine to be introduced and food as new information. For example, the extraction unit 12 refers to a database that stores data of interaction between medicine and food, and extracts information indicating interaction with food as new information. In a case where the medicine to be introduced is an antibacterial medicine, the extraction unit 12 may extract information about resistance of bacteria to the introduced antibacterial medicine as new information. For example, the extraction unit 12 refers to the data of the antibiogram of the antibacterial medicine, and extracts information about the resistance of the bacteria to the introduced antibacterial medicine as new information.

The extraction unit 12 may extract information about interaction as new information based on a structural formula of a medicine to be introduced and a structural formula of another medicine to be used for a treatment for an injury or disease. The extraction unit 12 estimates an interaction with another medicine from the structural formula of the medicine to be introduced using, for example, an interaction estimation model. The interaction estimation model is a machine learning model that estimates an interaction using a structural formula of a medicine to be estimated as an input. The interaction estimation model generates a graph with atoms as nodes and bonds between atoms as edges based on, for example, a structural formula of a medicine to be estimated. The interaction estimation model converts, for example, the generated graph into a feature amount. The interaction estimation model estimates an interaction between medicines based on, for example, similarity between feature amounts. The interaction estimation model is generated, for example, by learning the relationship between the structural formula of each medicine to interact and the interaction. The interaction estimation model is generated by deep learning using a neural network, for example. The machine learning algorithm for generating the interaction estimation model is not limited to the above. The interaction estimation model is generated, for example, in a learning means outside the data generation device 10. The interaction estimation model may be generated by a learning means (not illustrated) in the data generation device 10.

The extraction unit 12 may further extract information about a treatment for an injury or disease to which the introduced medical practice is applied from the data of the electronic medical record in the target medical institution. For example, the extraction unit 12 extracts information indicating an action taken in a target medical institution for an injury or disease to which the medical practice to be introduced is applied. For example, in a case where the medical practice to be introduced is an antibacterial medicine used at the time of surgery, the extraction unit 12 extracts, from the electronic medical record, information indicating an action taken for a patient regarding administration of an antibacterial medicine taken for surgery in a target medical institution.

The generation unit 13 generates, as time series medical information, information indicating a time series action to be taken for a patient regarding a medical practice to be introduced, based on the extracted new information. For example, the generation unit 13 generates time series medical information optimized for a medical practice to be introduced based on the extracted new information. The time series medical information is, for example, a clinical path.

For example, the generation unit 13 generates time series medical information using a language model based on the extracted new information. For example, the generation unit 13 generates the time series medical information by inputting a prompt including an instruction to generate the time series medical information and the new information to the language model. In a case where the time series medical information is the clinical path, the generation unit 13 generates the clinical path using, for example, a prompt including an instruction to generate the clinical path and new information as an input of the language model. For example, the generation unit 13 generates the clinical path by inputting, to the language model, a prompt for instructing generation of the clinical path reflecting the new information extracted from the predetermined medical information. The instruction to generate the clinical path may include the target name of the injury or disease to which the clinical path is applied.

The generation unit 13 may generate time series medical information related to a name of the injury or disease acquired by the acquisition unit 11. For example, the generation unit 13 generates time series medical information by inputting a prompt including an instruction to generate time series medical information related to a name of the injury or disease and new information to the language model. For example, in a case where the time series medical information is a clinical path, the generation unit 13 generates the clinical path by inputting a prompt including an instruction to generate the clinical path related to the name of the injury or disease and new information to the language model.

For example, a machine learning model also referred to as a large-scale language model can be used as the language model. For example, GPT-2, GPT-3, GPT-3.5, or GPT-4 can be used as the language model. Claude3, Claude3.5, T5, BERT, RoBERTa, or ELECTRA may be used as the language model. The language model used for the process of generating the time series medical information is not limited to the above. The language model used for the process of extracting the new information from the predetermined medical information and the language model used for the process of generating the time series medical information may be the same or different.

The generation unit 13 may generate the time series medical information using a language model that operates in an information processing device outside the data generation device 10. In a case of using a language model that operates in an external information processing device, the generation unit 13 outputs a prompt including an instruction to generate time series medical information and new information to the information processing device in which the language model operates, for example. For example, the generation unit 13 acquires the time series medical information generated by the language model from the information processing device in which the language model operates.

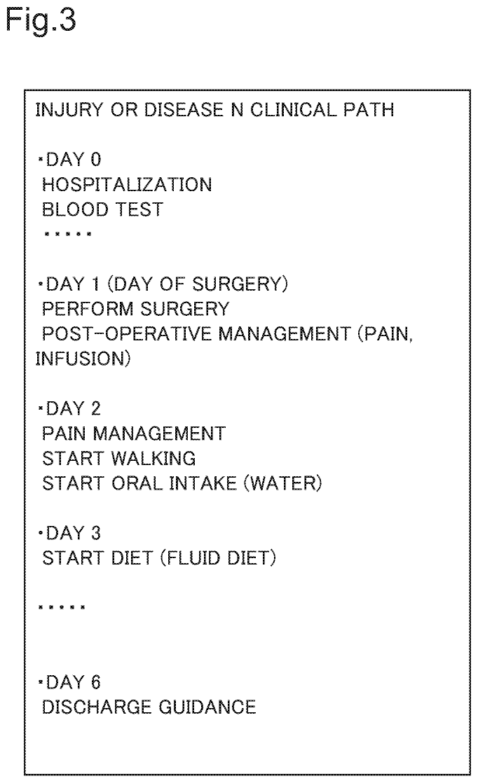

FIG. 3 is a diagram illustrating an example of a clinical path generated as time series medical information. The example of FIG. 3 is an example of a clinical path in a case where surgery related to the injury or disease N is performed. In the example of FIG. 3, for example, as a method of surgery for the injury or disease N, a clinical path is generated in order to newly introduce laparoscopic surgery. In a case where surgery that has been performed by laparotomy in a medical institution is performed by laparoscopic surgery, the action taken for a patient after surgery may change. In such a case, a clinical path for a newly introduced medical practice is generated. In the example of FIG. 3, the action taken by day 6 with the day of performing the surgery as day 1 is illustrated. In the example of FIG. 3, the action taken by the day before the surgery day is illustrated as the action on day 0. A medical practitioner in a medical institution determines an action to be taken for a patient according to the number of elapsed days from surgery, for example, with reference to a clinical path as illustrated in the example of FIG. 3.

For example, the generation unit 13 may generate the time series medical information reflecting the introduced medical practice using the language model based on the extracted new information and the time series medical information applied to a treatment for an injury or disease. For example, in a case of generating time series medical information Cb related to a medical practice Mb to be newly introduced, the generation unit 13 may generate a time series medical information Cb related to an introduced medical practice Mb based on time series medical information Ca applied to the medical practice Ma and new information extracted from predetermined information related to the newly introduced medical practice Mb. For example, in a case where an anticancer agent for colorectal cancer is newly introduced, the generation unit 13 generates time series medical information indicating an administration procedure of the anticancer agent to be newly introduced using a language model based on new information extracted from a package insert of the anticancer agent to be newly introduced and an administration procedure of the anticancer agent already used in a medical institution to be introduced.

For example, the generation unit 13 may generate the time series medical information reflecting the introduced medical practice using the language model based on the extracted new information, the information about the medical practice already applied to the treatment for the injury or disease in the introduced medical institution, and the time series medical information already applied to the treatment for the injury or disease. The information about the medical practice already applied to a treatment for an injury or disease in the introduced medical institution is, for example, information corresponding to new information in the newly introduced medical practice. For example, in a case where an anticancer agent for colorectal cancer is newly introduced, the generation unit 13 may generate a clinical path for the anticancer agent to be newly introduced using a language model based on a package insert of the anticancer agent to be newly introduced, a package insert of the anticancer agent already used in the medical institution to be introduced, and a clinical path for the anticancer agent already used.

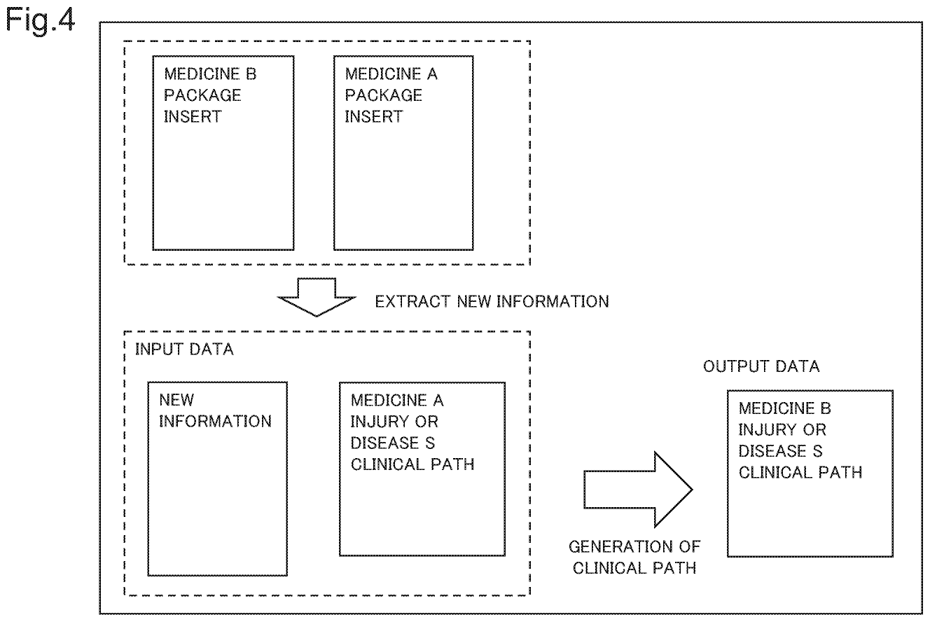

FIG. 4 is a diagram schematically illustrating an example of a process of generating time series medical information reflecting an introduced medical practice based on the extracted new information and the time series medical information applied to a treatment for an injury or disease. The example of FIG. 4 illustrates, for example, a process of generating a clinical path in a case where administration of a medicine B to a patient with an injury or disease S is newly started in a hospital where a medicine A has been administered to the patient with the injury or disease S. In the example of FIG. 4, the extraction unit 12 extracts, for example, a difference between information included in the package insert of the medicine A and information included in the package insert of the medicine B as new information. When the new information is extracted, for example, the generation unit 13 generates a clinical path in a case where the medicine B is used for treatment for the injury or disease S using the language model based on the extracted new information and the clinical path regarding treatment for the injury or disease S using the medicine A. In this case, for example, the generation unit 13 generates the clinical path for the medicine B by reflecting the new information, which is the difference between the information included in the package insert of the medicine A and the information included in the package insert of the medicine B, in the clinical path regarding the treatment for the injury or disease S using the medicine A using the language model. For example, the generation unit 13 replaces a portion related to a difference between the information included in the package insert of the medicine A and the information included in the package insert of the medicine B in the clinical path regarding the treatment for the injury or disease S using the medicine A with the information about the medicine B using the language model, thereby generating the clinical path in a case where the medicine B is used for the treatment for the injury or disease S.

The generation unit 13 may generate the time series medical information using a technique of retrieval-augmented generation (RAG). For example, the generation unit 13 generates the time series medical information by inputting the time series medical information used for the medical practice for a target that is the same as a target for which the medical practice to be introduced is performed into the language model as a reference document that is a document for reference.

For example, the generation unit 13 extracts, as a reference document, time series medical information used for a medical practice for a target that is the same as a target for which the medical practice to be introduced is performed. That the target is the same means, for example, that the injury or disease used for treatment is the same in the medical practice to be introduced. Same includes similar. The generation unit 13 may extract, as a reference document, time series medical information generated for a medical practice similar to the medical practice to be introduced. For example, the generation unit 13 generates the time series medical information by inputting the extracted reference document to the language model together with a prompt.

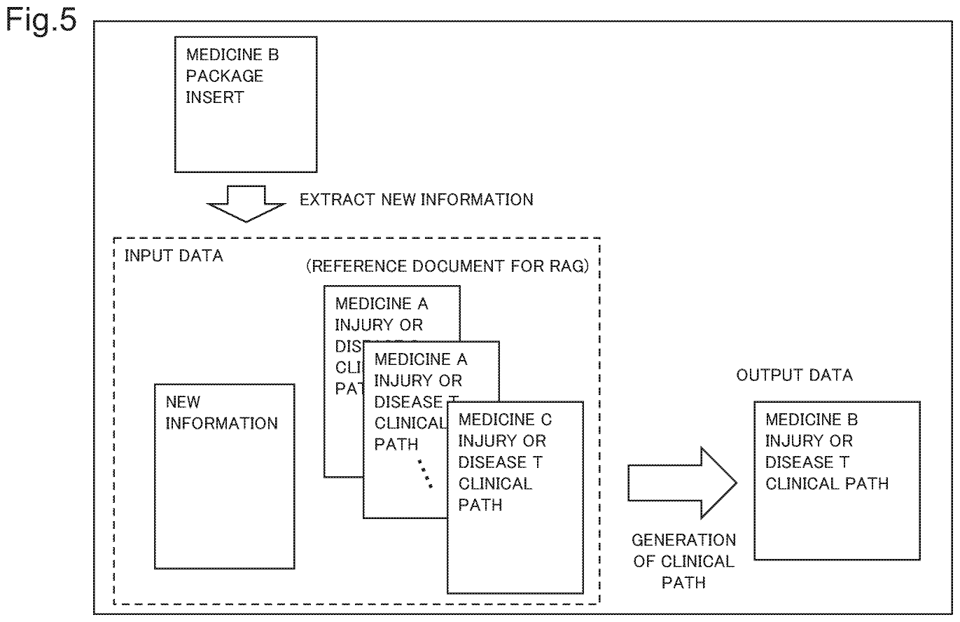

FIG. 5 is a diagram schematically illustrating an example of a process of generating time series medical information reflecting an introduced medical practice using the RAG method. The example of FIG. 5 illustrates a process of generating a clinical path as time series medical information in a case where a patient with an injury or disease T is treated using a medicine B, for example. In the example of FIG. 5, the extraction unit 12 extracts, for example, information about the administration method and side effects of the medicine B from the package insert of the medicine B as new information. When the new information is extracted, for example, the generation unit 13 generates a clinical path in a case where the medicine B is used for the treatment for the injury or disease T using the language model with the extracted new information as an input with a clinical path for the injury or disease and the medicine similar to at least one of the injury or disease T and the medicine B as a reference document.

The generation unit 13 may generate time series medical information including cautions regarding the condition of the patient extracted by the extraction unit 12. For example, in a case where a side effect that may occur at the timing of the time point Ta, the time point Tb, and the time point Tc when a medicine is administered to a patient is extracted as new information, the generation unit 13 generates information in which the side effects that may occur are disposed in time series order as the time series medical information.

The generation unit 13 may generate the time series medical information based on the new information and the information about the treatment in the injury or disease to which the medical practice to be introduced is applied. For example, the generation unit 13 generates time series medical information based on information indicating an action taken in a target medical institution in an injury or disease to which the medical practice to be introduced is applied. The generation unit 13 generates the time series medical information by reflecting the new information in the information indicating the action taken in the target medical institution in the injury or disease to which the medical practice to be introduced is applied.

The generation unit 13 may generate time series medical information having a branch. The generation unit 13 generates, for example, time series medical information related to a variance. The variance refers to, for example, a state in which a patient's symptom is out of an assumed symptom range. For example, the generation unit 13 generates time series medical information related to the variance based on cautions regarding the condition of the patient caused by performing a medical practice on the patient, the cautions being extracted as the new information. The generation unit 13 generates, as time series medical information related to the variance, the content of an action to be taken for a patient in a case where a condition of the patient indicated in the cautions regarding the condition of the patient occurs. For example, the generation unit 13 generates time series medical information related to the variance based on information about an action taken in a case where a similar symptom occurs in an existing medical practice.

The generation unit 13 may generate the time series medical information based on a constraint in the target medical institution. For example, the generation unit 13 generates time series medical information so that the treatment and the test using a medical device owned by a target medical institution are performed. The generation unit 13 generates time series medical information so that treatment is performed by a treatment method that can be handled by a target medical institution, for example. That is, for example, the generation unit 13 generates time series medical information that does not include a treatment and a test that cannot be performed by the target medical institution. For example, in a case where a brain examination is performed in a medical institution that has computed tomography (CT) but does not have magnetic resonance imaging (MRI), the generation unit 13 may generate time series medical information in such a way that the brain examination is performed using CT. For example, in a case where a stomach operation is performed in a medical institution in which the stomach operation can be performed by laparotomy but cannot be performed by laparoscopic surgery, the generation unit 13 may generate the time series medical information in such a way that the stomach operation is performed by the laparotomy.

The generation unit 13 may generate time series medical information according to a person who views the information. In a case where the time series medical information is generated using the language model, the generation unit 13 generates the time series medical information related to the person who views the information, for example, by inputting a prompt including the information of the person who views the information into the language model. For example, in a case where the person who views the information is a medical practitioner, the generation unit 13 generates time series medical information according to the role of the person who views the information. The medical practitioner is, for example, a doctor, a nurse, a pharmacist, a clinical laboratory technician, a physical therapist, a dietitian, or a counselor. The medical practitioner is not limited to the above. In a case where the person who views the information is a nurse among the medical practitioners, the generation unit 13 generates, for example, time series medical information about an action taken by the nurse for the patient. For example, in a case where the person who views the information is a patient, the generation unit 13 generates time series medical information using a term for the patient. In a case where the person who views the information is a patient, the generation unit 13 may generate time series medical information in which expression indicating the degree of symptoms caused by medical practice for the patient is suppressed.

The generation unit 13 may generate time series medical information indicating the content of an action for the patient by each of the persons having a plurality of respective roles. The generation unit 13 generates, for example, time series medical information indicating the content of an action to be taken for a patient by each of the persons having a plurality of respective roles at each stage of a treatment. For example, the generation unit 13 generates time series medical information indicating the content of an action to be taken for a patient by each of a doctor and a nurse at each stage of a treatment.

The output unit 14 outputs the generated time series medical information. The output unit 14 outputs the generated time series medical information to the terminal device 20, for example. The output unit 14 may output the time series medical information by emphasizing a characteristic point in the medical practice to be introduced. For example, the output unit 14 outputs the time series medical information by emphasizing a portion changed from the already introduced medical practice in the medical practice to be introduced. The output unit 14 may output an action that requires attention regarding the condition of the patient among actions for the patient included in the time series medical information in an emphasized manner. The output unit 14 outputs the time series medical information so that, for example, an emphasized portion is displayed in a color different from colors of other portions. An aspect of emphasizing part of the time series medical information can be appropriately set.

The storage unit 15 stores, for example, data related to a process of generating time series medical information. The storage unit 15 stores, for example, predetermined medical information. The storage unit 15 stores, for example, new information extracted from predetermined medical information. The storage unit 15 stores, for example, the generated time series medical information. The storage unit 15 stores, for example, a reference document. The storage unit 15 stores, for example, a language model used for extracting new information. The storage unit 15 stores, for example, a language model used for generation of time series medical information. The language model may be stored in a storage means outside the data generation device 10. The storage unit 15 stores, for example, an extraction model. The storage unit 15 stores, for example, an interaction estimation model. The extraction model and the interaction estimation model may be stored in a storage means outside the data generation device 10.

The terminal device 20 is, for example, an information processing device used by a person in charge in a process of generating time series medical information indicating a time series action taken for a patient regarding a medical practice to be introduced. The person in charge is, for example, a person who creates time series medical information or a person who performs the medical practice on a patient using time series medical information. The person in charge is, for example, a medical practitioner. The medical practitioner is, for example, a doctor, a nurse, a pharmacist, a clinical laboratory technician, a physical therapist, a dietitian, or a counselor. The medical practitioner is not limited to the above. The person in charge is not limited to the above. The terminal device 20 acquires the time series medical information from the output unit 14 of the data generation device 10, for example. The terminal device 20 outputs the time series medical information to a display device (not illustrated), for example.

As the terminal device 20, for example, a personal computer, a tablet computer, a smartphone, or a smartwatch can be used. The information processing device used for the terminal device 20 is not limited to the above.

The data management device 30 is, for example, an information processing device that stores predetermined medical information. The data management device 30 acquires, for example, cautions, usage, and application criteria regarding a medicine, a medical device, a therapeutic method, or a surgical method to be newly introduced into a medical institution as predetermined medical information. For example, the data management device 30 may acquire predetermined medical information from an information processing device that provides predetermined medical information connected via a network. The predetermined medical information is not limited to the above.

The data management device 30 may store data related to the treatment of a patient in a medical institution. The data regarding the treatment of the patient is, for example, data of an electronic medical record input by a doctor. The information in the electronic medical record may be data input by a nurse, a laboratory technician, a physical therapist, or a counselor. The data regarding the treatment of the patient may be information of one or more items of patient disease information, complication information, biomarkers, disease statuses, guideline scores, medical histories, efficacy, and test results other than data described in the electronic medical record. The data management device 30 outputs data regarding the treatment to the acquisition unit 11 of the data generation device 10, for example.

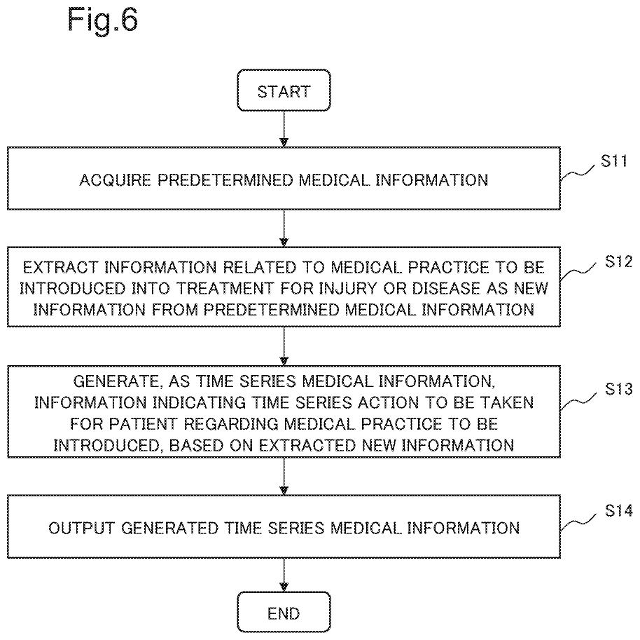

An operation of the data generation device 10 in a process of generating time series medical information indicating a time series action taken for a patient regarding a medical practice to be introduced will be described. FIG. 6 is an example of a flow in a process in which the data generation device 10 generates time series medical information indicating a time series action taken for a patient regarding a medical practice to be introduced.

The acquisition unit 11 acquires, for example, predetermined medical information (step S11). The acquisition unit 11 acquires predetermined medical information from the data management device 30, for example.

When the predetermined medical information is acquired, the extraction unit 12 extracts information about a medical practice to be introduced into a treatment for an injury or disease from the predetermined medical information as new information (step S12).

When the new information is extracted, the generation unit 13 generates, as the time series medical information, information indicating a time series action taken for the patient regarding the medical practice to be introduced based on the extracted new information (step S13).

When the time series medical information is generated, the output unit 14 outputs the generated time series medical information (step S14). The output unit 14 outputs the generated time series medical information to the terminal device 20, for example.

Each process in the data generation device 10 may be executed in a distributed manner in a plurality of information processing devices connected via a network. For example, the process in the extraction unit 12 and the process in the generation unit 13 may be performed by different information processing devices. Which information processing device performs each process in the data generation device 10 can be appropriately set.

The data generation device 10 extracts information about a medical practice to be introduced into a treatment for an injury or disease from predetermined medical information as new information. Based on the extracted new information, the data generation device 10 generates, as time series medical information, information indicating a time series action to be taken for a patient regarding a medical practice to be introduced. In this manner, by generating the information indicating a time series action to be taken for the patient regarding the medical practice to be introduced, the data generation device 10 can easily generate, for example, the information indicating the action to be taken for the patient regarding the medical practice to be introduced. By generating the time series medical information indicating the action to be taken for the patient regarding the medical practice to be introduced, the data generation device 10 can support decision making regarding the treatment of the patient, for example.

By generating the time series medical information according to the person who views, the data generation device 10 can present the time series medical information that is easy for the person who views the information to understand, for example. By generating the time series medical information based on the difference between the medical practice to be introduced and the existing medical practice, the medical practitioner who views the time series medical information can easily cope with the medical practice to be newly introduced while utilizing the experience in the medical practice that has been performed so far. By generating the time series medical information including cautions regarding the condition of the patient due to performing the medical practice to be introduced, the medical practitioner who views the time series medical information can grasp, for example, an action that requires new attention regarding the condition of the patient. Therefore, for example, the data generation device 10 can generate highly effective time series medical information for a medical practice to be newly introduced.

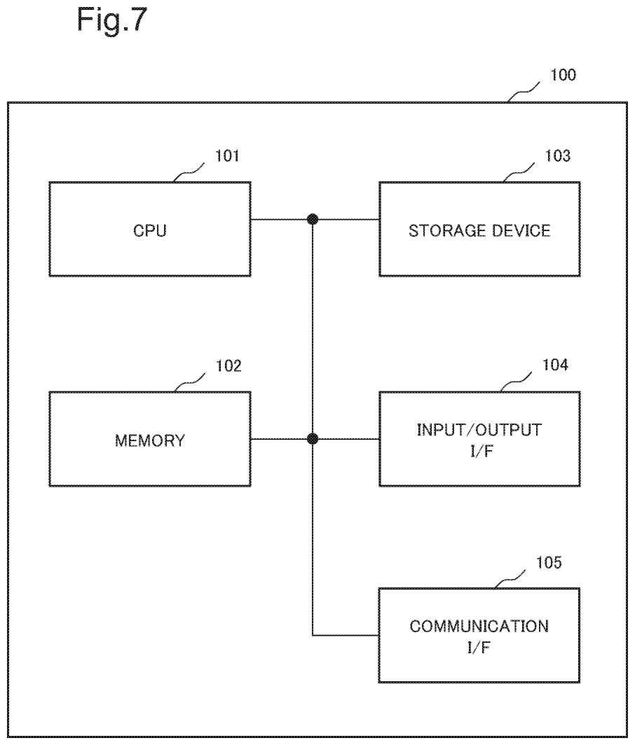

Each process in the data generation device 10 can be implemented by executing a computer program on a computer. FIG. 7 illustrates an example of a configuration of a computer 100 that executes a computer program for executing each process in the data generation device 10. The computer 100 includes a central processing unit (CPU) 101, a memory 102, a storage device 103, an input/output interface (I/F) 104, and a communication I/F 105.

The CPU 101 reads and executes a computer program for executing each process from the storage device 103. The CPU 101 may be configured by a combination of a plurality of CPUs. The CPU 101 may be configured by a combination of a CPU and another type of processor. For example, the CPU 101 may be configured by a combination of a CPU and a graphics processing unit (GPU). The memory 102 includes a dynamic random access memory (DRAM) or the like, and temporarily stores a computer program executed by the CPU 101 and data being processed. The storage device 103 stores a computer program executed by the CPU 101. The storage device 103 includes, for example, a nonvolatile semiconductor storage device. As the storage device 103, another storage device such as a hard disk drive may be used. The input/output I/F 104 is an interface that receives an input from an operator and outputs display data and the like. The communication I/F 105 is an interface that transmits and receives data to and from the terminal device 20 and another information processing device. The terminal device 20 can also have a configuration similar to that of the computer 100.

The computer program used for executing each process can also be distributed by being stored in a computer-readable recording medium that non-transitory records data. As the recording medium, for example, a magnetic tape for data recording or a magnetic disk such as a hard disk can be used. As the recording medium, an optical disk such as a compact disc read only memory (CD-ROM) can also be used. A nonvolatile semiconductor storage device may be used as a recording medium.

In a medical institution, time series information indicating a plan of a treatment, a test, and the like performed on a patient may be used. For example, a clinical path is used as time series information indicating a plan of a treatment, a test, and the like performed on a patient. A medical practitioner in a medical institution refers to, for example, a clinical path, and takes an action related to a treatment, a test, and the like for a patient at a timing described in the clinical path. The clinical path is created, for example, for each injury or disease. An information system that supports the creation may be used to create the clinical path.

The clinical path creation support device of JP 2015-197733 A narrows down the candidates of the event that can be executed for the patient based on the name and attribute of the injury or disease of the patient. The clinical path creation support device of JP 2015-197733 A displays event candidates on a clinical path creation screen.

In order to solve the above problems, an object of the present disclosure is to provide a data generation device and the like capable of easily generating information indicating an action to be taken for a patient in a medical practice to be introduced in time series.

According to the present disclosure, it is possible to easily generate information indicating an action to be taken for a patient in a medical practice to be introduced in time series.

Some or all of the above-described example embodiments may be described as the following Supplementary Notes, but are not limited to the following.

### Supplementary Note 1

A data generation device including
an extraction unit that extracts information about a medical practice to be introduced into a treatment for an injury or disease from predetermined medical information as new information,
a generation unit that generates, as time series medical information, information indicating a time series action to be taken for a patient regarding a medical practice to be introduced based on the extracted new information, and
an output unit that outputs the generated time series medical information.

### Supplementary Note 2

The data generation device according to Supplementary Note 1, wherein
the generation unit generates the time series medical information using a language model based on the extracted new information.

### Supplementary Note 3

The data generation device according to Supplementary Note 2, wherein
the generation unit generates time series medical information reflecting an introduced medical practice using the language model based on the extracted new information and time series medical information applied to a treatment for an injury or disease.

### Supplementary Note 4

The data generation device according to any one of supplementary Notes 1 to 3, wherein
the extraction unit extracts, as the new information, a caution regarding a condition of a patient, the caution being caused by performing a medical practice on the patient, from the predetermined medical information, and
the generation unit generates the time series medical information including the extracted caution regarding the condition of the patient.

### Supplementary Note 5

The data generation device according to any one of Supplementary Notes 1 to 3, wherein
the extraction unit extracts, as the new information, information about an interaction between an introduced medicine and another medicine used for a treatment for an injury or disease.

### Supplementary Note 6

The data generation device according to any one of Supplementary Notes 1 to 3, wherein
the extraction unit further extracts information about a treatment for an injury or disease to which a medical practice to be introduced is applied from data of an electronic medical record in a target medical institution, and
the generation unit generates the time series medical information based on the extracted new information and information about a treatment for an injury or disease to which the medical practice to be introduced is applied.

### Supplementary Note 7

The data generation device according to any one of Supplementary Notes 1 to 3, wherein the generation unit generates the time series medical information having a branch.

### Supplementary Note 8

The data generation device according to any one of Supplementary Notes 1 to 3, wherein
the time series medical information is a clinical path.

### Supplementary Note 9

The data generation device according to Supplementary Note 5, wherein
the extraction unit extracts information about the interaction as the new information based on a structural formula of a medicine to be introduced and a structural formula of another medicine used for a treatment for an injury or disease.

### Supplementary Note 10

The data generation device according to Supplementary Note 9, wherein
the extraction unit extracts information about the interaction as the new information by estimating an interaction between a medicine to be introduced and another medicine used for a treatment for an injury or disease using a machine learning model for estimating an interaction with a structural formula of a medicine to be estimated as an input.

### Supplementary Note 11

The data generation device according to any one of Supplementary Notes 1 to 3, wherein
the generation unit generates the time series medical information based on a constraint in a target medical institution.

### Supplementary Note 12

The data generation device according to any one of Supplementary Notes 1 to 3, wherein
the generation unit generates the time series medical information according to a person who views the information.

### Supplementary Note 13

The data generation device according to any one of Supplementary Notes 1 to 3, wherein
the output unit outputs a treatment requiring attention among actions for a patient included in the time series medical information in a highlighted manner.

### Supplementary Note 14

The data generation device according to Supplementary Note 2, wherein
the extraction unit extracts the new information from the predetermined medical information using a language model.

### Supplementary Note 15

A data generation method including
extracting information about a medical practice introduced into a treatment for an injury or disease from predetermined medical information as new information,
generating, as time series medical information, information indicating a time series action to be taken for a patient regarding a medical practice to be introduced based on the extracted new information, and
outputting the generated time series medical information.

### Supplementary Note 16

A non-transitory recording medium that records a program for causing a computer to execute the steps of
extracting information about a medical practice introduced into a treatment for an injury or disease from predetermined medical information as new information,
generating, as time series medical information, information indicating a time series action to be taken for a patient regarding a medical practice to be introduced based on the extracted new information, and
outputting the generated time series medical information.

Some or all of the configurations described in Supplementary Notes 2 to 14 dependent on the above-described Supplementary Note 1 can also depend on Supplementary Note 15 and Supplementary Note 16 by the same dependency relationship as Supplementary Notes 2 to 14. Furthermore, some or all of the configurations described as the Supplementary Notes can be similarly dependent on not only the Supplementary Notes 1, 15, and 16, but also various pieces of hardware and software, and various recording means or systems for recording software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents. Further, it is noted that the inventor's intent is to retain all equivalents of the claimed invention even if the claims are amended during prosecution.

1. A data generation device comprising:
at least one memory storing instructions; and
at least one processor configured to access the at least one memory and execute the instructions to:
extract information about a medical practice to be introduced into a treatment for an injury or disease from predetermined medical information as new information, and information about a treatment for an injury or disease to which a medical practice to be introduced is applied from data of an electronic medical record in a target medical institution;
generate, as time series medical information, information indicating a time series action to be taken for a patient regarding a medical practice to be introduced, by using a language model, based on the extracted new information and information about a treatment for an injury or disease to which the medical practice to be introduced is applied; and
output the generated time series medical information.

2. The data generation device according to claim 1, wherein
the at least one processor is further configured to execute the instructions to:
generate the time series medical information reflecting an introduced medical practice, based on the extracted new information and time series medical information applied to a treatment for an injury or disease.

3. The data generation device according to claim 1, wherein
the at least one processor is further configured to execute the instructions to:
extract, as the new information, a caution regarding a condition of a patient, the caution being caused by performing a medical practice on the patient, from the predetermined medical information; and
generate the time series medical information including the extracted caution regarding the condition of the patient.

4. The data generation device according to claim 1, wherein
the at least one processor is further configured to execute the instructions to:
extract, as the new information, information about an interaction between an introduced medicine and another medicine used for a treatment for an injury or disease.

5. The data generation device according to claim 1, wherein
the at least one processor is further configured to execute the instructions to:
generate the time series medical information having a branch.

6. The data generation device according to claim 1, wherein the time series medical information is a clinical path.

7. The data generation device according to claim 4, wherein
the at least one processor is further configured to execute the instructions to:
extract information about the interaction as the new information based on a structural formula of a medicine to be introduced and a structural formula of another medicine used for a treatment for an injury or disease.

8. The data generation device according to claim 7, wherein
the at least one processor is further configured to execute the instructions to:
extract information about the interaction as the new information by estimating an interaction between a medicine to be introduced and another medicine used for a treatment for an injury or disease using a machine learning model for estimating an interaction with a structural formula of a medicine to be estimated as an input.

9. The data generation device according to claim 1, wherein
the at least one processor is further configured to execute the instructions to:
generate the time series medical information based on a constraint in a target medical institution.

10. The data generation device according to claim 1, wherein
the at least one processor is further configured to execute the instructions to:
generate the time series medical information according to a person who views the information.

11. The data generation device according to claim 1, wherein
the at least one processor is further configured to execute the instructions to:
output a treatment requiring attention among actions for a patient included in the time series medical information in a highlighted manner.

12. The data generation device according to claim 1, wherein
the at least one processor is further configured to execute the instructions to:
extract the new information from the predetermined medical information by using a language model.

13. A data generation method comprising:
extracting information about a medical practice to be introduced into a treatment for an injury or disease from predetermined medical information as new information, and information about a treatment for an injury or disease to which a medical practice to be introduced is applied from data of an electronic medical record in a target medical institution;
generating, as time series medical information, information indicating a time series action to be taken for a patient regarding a medical practice to be introduced, by using a language model, based on the extracted new information and information about a treatment for an injury or disease to which the medical practice to be introduced is applied; and
outputting the generated time series medical information.

14. The data generation method according to claim 13, the data generation method further comprising:
generating the time series medical information reflecting an introduced medical practice, based on the extracted new information and time series medical information applied to a treatment for an injury or disease.

15. The data generation method according to claim 13, the data generation method further comprising:
extracting, as the new information, a caution regarding a condition of a patient, the caution being caused by performing a medical practice on the patient, from the predetermined medical information; and
generating the time series medical information including the extracted caution regarding the condition of the patient.

16. The data generation method according to claim 13, the data generation method further comprising:
extracting, as the new information, information about an interaction between an introduced medicine and another medicine used for a treatment for an injury or disease.

17. The data generation method according to claim 13, the data generation method further comprising:
generating the time series medical information having a branch.

18. The data generation method according to claim 13, wherein the time series medical information is a clinical path.

19. The data generation method according to claim 16, the data generation method further comprising:
extract information about the interaction as the new information based on a structural formula of a medicine to be introduced and a structural formula of another medicine used for a treatment for an injury or disease.

20. A non-transitory recording medium that records a program for causing a computer to execute the steps of:
extracting information about a medical practice to be introduced into a treatment for an injury or disease from predetermined medical information as new information, and information about a treatment for an injury or disease to which a medical practice to be introduced is applied from data of an electronic medical record in a target medical institution;
generating, as time series medical information, information indicating a time series action to be taken for a patient regarding a medical practice to be introduced, by using a language model, based on the extracted new information and information about a treatment for an injury or disease to which the medical practice to be introduced is applied; and
outputting the generated time series medical information.
